(19) United States
(12) Patent Application Publication
(10) Pub. No.: US 2020/0147037 A1
(43) Pub. Date: May 14, 2020

(54) NEW TREATMENT OF BRAIN CANCER

(71) Applicant: CORAL BIOME, MARSEILLE CEDEX 09 (FR)

(72) Inventor: Sandrine COURTES, MARSEILLE (FR)

(21) Appl. No.: 16/619,524

(22) PCT Filed: Jun. 5, 2018
(86) PCT No.: PCT/EP2018/000288

§ 371 Date: Dec. 5, 2019

(30) Foreign Application Priority Data
Jun. 5, 2017 (EP) 17000937.7

(51) Int. Cl. A61K 31/357; A61K 35/614; A61P 35/00; A61K 9/00

(52) U.S. Cl. CPC A61K 31/357; A61K 35/614; A61K 31/175; A61K 9/0019; A61P 35/00

(57) ABSTRACT

The present invention is directed to a composition comprising palytoxin (PLTX), an analogue or a derivative thereof for use by a non-intracerebral or an intracerebral administration in the prevention and/or in the treatment of a subject suffering from a brain tumor.

### FIELD OF THE INVENTION

The invention relates to the field of cancer therapy and in particular to brain tumor treatment and prevention.

### DESCRIPTION OF PRIOR ART

According to the 2016 World Health Organization Classification of Tumors of the Central Nervous System, more than 120 different types of brain and spinal cord tumors exist, which tumors can affect both adults and children. Thus, brain tumors are the leading cause of solid cancer death in children under the age of 20 and they are the third leading cause of solid cancer death in young adults ages 20-39. Gliomas account for more than 74% of malignant brain tumors, and of these, glioblastoma is the most frequent (60-75% of all astrocytomas) and most malignant histologic type. The prognosis of glioma patients is still poor. With standard treatment, median survival for adults with an anaplastic astrocytoma is about two to three years. For adults with more aggressive glioblastoma, treated with concurrent temozolomide and radiation therapy subsequent to tumor removal by surgery, median survival is about 14.6 months and two-year survival is 30%, while fewer than 3% of glioblastoma patients are still alive at 5 years after diagnosis. Malignant brain primary tumors and mostly glioblastomas, thus represent a therapeutic challenge in urgent need of new innovative treatment options.

Among the brain tumors, primary brain tumors (originating in the brain) occur in 8 out of 100,000 people. Now, when also considering secondary brain tumors—i.e. cancer originating in other areas of the body that has spread to the brain by metastasis—, this number rises to approximately 32 per 100,000. In fact, in adults, brain metastasis are the most common intracranial tumors, and are 10 times more common than primary malignant CNS tumors, with at least 170 000 new cases reported in the United States each year. Main cancers leading to brain metastasis are lung and breast cancers, for a third and a quarter of reported cases respectively, followed by melanomas, cancers of the digestive tract, renal cancer and lymphoma. Consequently, CNS metastasis significantly outnumbers primary brain tumors, and carries a bad prognosis.

It exists only limited treatment strategies for managing sanctuary diseases in the brain. Despite the use of targeted therapies, particularly radiosurgery, which has broadened therapeutic options for CNS metastases, patients still respond minimally to treatments and their prognosis remains poor. Complete removal by surgery is unlikely due to tumor infiltration, patient relapsing after 1 rst line treatment can difficulty be retreated by radiotherapy because of radiation side effects. Still today, many patients with CNS involvement die from neurological progression despite controlled systemic disease.

The brain is considered as a tumor sanctuary, because its micro-environment protects tumor cells from chemotherapies through various signals stimulating proliferation and survival. Many drugs efficient on brain cancer cells in vitro, fail to be efficient in ex vivo 3D models, in vivo and clinically. The brain endogenous growth factor, as the endothelial and astrocytic compartments play a major role in this pro-tumoral micro-environment (R. Langley et al. Clinical Chemistry 59:1, 180-189 2013). Therefore, all brain cancers, primary and secondary, (issued from metastatic peripheral cancers) tend to be highly invasive, associated to very poor prognosis and in urgent need of innovative molecules able to overcome this pro-tumoral micro-environment effect.

Poorer therapeutic outcomes are also mostly linked to the inability of many systemic chemotherapeutic agents to penetrate the blood-brain barrier (BBB), which represents a major hindrance in the successful delivery of CNS drugs to the brain. In fact, more than 98% of all small molecules and almost 100% of large molecules cannot cross the blood-brain barrier (BBB). Although biologics such as proteins, peptides, genes and oligonucleotides are promising new drugs for treating CNS tumors, their transport across the BBB from the systemic circulation is often very much restricted. Thus, such failure of active therapeutic agents to overcome this natural first line of defense of the brain represented by the blood-brain barrier (BBB) has allowed brain tumors and metastases to become a burgeoning clinical challenge.

Accordingly, there is still an urgent need for new treatments against brain tumors, which can overcome the limitations encountered in this field.

The invention aims to meet the afore mentioned needs.

### SUMMARY OF THE INVENTION

By testing the anti-tumoral effect of palytoxin (PLTX) directly administered intracranially in a brain tumor model, the inventors observed that their “negative” control corresponding to an intraperitoneal administration shows a very strong therapeutic benefit.

This unexpected observation established that palytoxin (PLTX) given intraperitoneally can penetrate the central nervous system (CNS) through the blood brain barrier (BBB) and overcome the intracerebral microenvironment effect so as to inhibit tumor progression in the brain in vivo in an incurable tumor model.

Accordingly, a first object of the invention is directed to a composition comprising palytoxin (PLTX), an analogue or a derivative thereof for use in the prevention and/or in the treatment of a subject suffering from a brain tumor by a non-intracerebral administration.

In a second object, the invention is directed to a method for preventing and/or treating a subject suffering from a brain tumor comprising the administration to said subject of an effective amount of a composition comprising palytoxin (PLTX), an analogue or a derivative thereof, wherein said administration is a non-cerebral administration.

### BRIEF DESCRIPTION OF THE DRAWINGS

FIG. 1 shows the palytoxin IC50 at 72 h from different origins on U-87 MG, U-118, A-375 and A-549 cell lines.

FIG. 2 shows the survival curve of mice from a brain tumor model following palytoxin intracerebral and intraperitoneal injection

FIG. 3 shows the weight curve of mice with PLTX intraperitoneal injection compared with DMSO intracerebral injection.

FIGS. 4 show the evolution of a human glioblastoma in a mouse model following DMSO (A), PLTX intracerebral (B) and PLTX intraperitoneal (C) administration. The outline of the tumor is indicated by a line hatched.

FIG. 5 shows the results of in-vivo treatment of brain metastases from human lung cancer by DMSO, PACLITAXEL or PALYTOXIN (PLTX)

FIGS. 6 shows ex-vivo PLTX treatment of human glioblastoma biopsies

### DETAILED DESCRIPTION

In a first aspect, the invention relates to a composition comprising palytoxin (PLTX), an analogue or a derivative thereof for use by a non-intracerebral administration in the prevention and/or in the treatment of a subject suffering from a brain tumor.

As used herein, a subject refers to an animal, preferably to a mammal, which is an adult or a juvenile. Preferably, said subject is a human, and may be an adult or a child.

Palytoxin (PLTX) refers to a toxin originally isolated in 1971. This non-protein compound corresponds to a fatty alcohol, and is produced by several marine species or corals species. Palytoxin (PLTX) targets the sodium-potassium pump protein by locking it into a position where it allows passive transport of both sodium and potassium ions, thereby destroying the ion gradient that is essential for most cells. The palytoxin (PLTX) chemical structure was published in 1982, and is as follows:

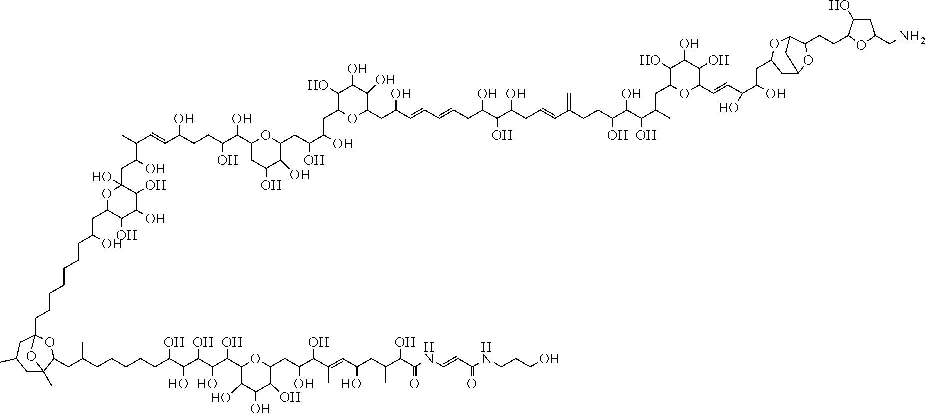 [SMILES: C=C(/C=C/CC(O)C(O)C(O)C/C=C/C=C/C(O)CC1OC(CC(O)C(O)CC2OC(C(O)C(O)CCC(O)/C=C/C(C)C(O)CC3(O)OC(CC(O)CCCCCCCC45CC(C)CC(C)(O4)C(CC(C)CCCCCC(O)C(O)C(O)C(O)C(O)C4OC(CC(O)C(O)/C(C)=C/C(O)CC(C)C(O)C(=O)N/C=C/C(=O)NCCCO)C(O)C(O)C4O)O5)C(O)C(O)C3O)CC(O)C2O)C(O)C(O)C1O)CCC(O)C(O)C(O)C(C)CC1OC(/C=C/C(O)C(O)CC2CC3CC(O2)C(CCC2OC(CN)CC2O)O3)C(O)C(O)C1O]

The PLTX structure is very complex with a long polyhydroxylated and partially unsaturated aliphatic backbone containing 64 chiral centers. PLTX is heat-stable, not inactivated by boiling, and stable in neutral aqueous solutions for prolonged periods. In contrast, a rapid degradation occurs under acid or alkaline conditions, leading to loss of its toxicity.

Preferably, palytoxin (PLTX) is purified or extracted from an Anthozoa, preferably from a Cnidaria, and still preferably from a Metazoa. Advantageously, palytoxin (PLTX) is purified or extracted from a coral, preferably from a Palythoa sp., and most preferably from Palythoa Heliodiscus.

As used herein “palytoxin (PLTX) analogue” refers to compounds having a similar backbone and produced in marine species corals species. As an example of palytoxin analogue, one can cite PLTX-b, Homo-PLTX, Bishomo-PLTX, Neo-PLTX, Deoxy-PLTX, 42-Hydroxy-PLTX, Mascarenotoxin-a, Mascarenotoxin-b, Mascarenotoxin-c, Ostreocin-d, Ovatoxin-a, Ovatoxin-b, Ovatoxin-c, Ovatoxin-d, Ovatoxin-e, Ovatoxin-f, Ovatoxin-g, Ovatoxin-h, and newly described palytoxin analogs.

As used herein “palytoxin (PLTX) derivative” refers to compounds having the palytoxin structure, as defined previously, with at least one, two or three distinct chemical functions, and/or to stereoisomers thereof and mixtures of such stereoisomers. Also, a palytoxin derivative may correspond to a compound obtained by palytoxin fragmentation, by palytoxin conjugation (e.g. antibodies, polypeptides, peptides, oligonucleotides, etc.) or by chemical synthesis, including but not limited to organic synthesis, hemisynthesis.

The composition of the invention may comprise a further anti-tumoral agent, such as standard anti-tumoral agents used for treating brain tumors like Temozolomide (e.g TEMODAL®), Carmustine (e.g. BiCNU®, GLIADEL®) or Cytarabine (DEPOCYTE).

While the invention corresponds to palytoxin non-intracerebral administration, the inventors have also established the interesting anti-tumoral effect obtained by intracerebral administration of palytoxin.

Accordingly, another object of the invention may be directed to a composition comprising palytoxin (PLTX), an analogue or a derivative thereof for use in the prevention and/or in the treatment of a subject suffering from a brain tumor by an intracerebral administration, and optionally by an intracerebroventricular administration, by an intra-thecal or by an intra-brain tumoral administration.

As used herein a brain tumor refers to a tumor located within the central nervous system as defined by the space surrounded by the blood brain barrier (BBB), the Blood-cerebrospinal fluid barrier (BCSFB) or the blood-peripheral nerves and ganglia barrier system. A brain tumor is also a combination of specific brain tissue and micro-environment constituting tumor sanctuaries.

The brain tumors comprise primary tumors starting within the brain, and secondary tumors that have spread from somewhere else, also known as brain metastasis tumors.

In a first preferred embodiment, the composition is for use in the prevention and/or in the treatment of a subject suffering from a primary brain tumor.

As defined by, the 2016 World. Health Organization Classification of Tumors of the Central Nervous System, primary brain tumors comprise: diffuse astrocytic and oligodendroglial tumors, other astrocytic tumors (astrocytomas) and other gliomas (astroblastomas); neuronal and mixed neuronal-glial tumors mainly anaplastic ganglioglioma; ependymal tumors (ependymomas); embryonal tumors; meningiomas and melanocytic tumors of the CNS; lymphomas of the CNS; histiocytic tumors of the CNS and Germ cell tumors of the CNS.

Diffuse astrocytic and oligodendroglial tumors comprise diffuse and anaplastic astrocytomas, glioblastomas, diffuse midline glioma, oligodendroglioma, anaplastic oligodendroglioma, oligoastrocytoma and anaplastic oligoastrocytomas.

Embryonal tumors comprise medulloblastomas, embryonal tumors with multi-layered rosettes, medulloepithelioma, CNS neuroblastoma, CNS ganglioneuroblastoma, CNS embryonal tumour NOS, atypical teratoid rhabdoid tumour and CNS embryonal tumour with rhabdoid features.

Meningiomas and melanocytic tumors of the CNS comprise mainly papillary meningioma, rhabdoid meningioma, anaplastic meningiomas, meningeal melanoma and meningeal melanomatosis.

The 2016 World Health Organization Classification does not anymore use the terms PNETs (Primitive NeuroEctodermal Tumor), and non-Hodgkin lymphomas which are included in the above list under the new terminology.

Preferably, said primary brain tumor is selected among diffuse astrocytic and oligodendroglial tumors and embryonal tumors, and most preferably said primary brain tumor is a glioblastoma.

In a second preferred embodiment, the composition is for use in the prevention and/or in the treatment of a subject suffering from a secondary brain tumor.

Secondary brain tumors comprise brain metastasis issued from lung, breast (mostly HER-2 positive breast cancer patients who have as high as a 30% incidence of brain metastasis), melanoma, digestive tract (e.g. colon), renal, lymphoma, pancreatic and prostate cancer, preferably from grade III or IV cancer, and most preferably from grade IV cancer.

Still preferably, secondary brains tumors comprise brain metastases issued from lung cancers, breast cancers and melanomas.

As used herein a non-intracerebral administration encompasses all the administrations with the exception of intracerebral administration, and preferably with the further exception of intrathecal administration and/or of intra-tumoral administration.

Advantageously, a non-intracerebral administration is selected in the group comprising oral, mucosal, intravenous, intramuscular, intradermal, subcutaneous, transdermal, and intraperitoneal administration, preferably a non-intracerebral administration is selected in the group comprising intraperitoneal, intravenous, and subcutaneous administration, and most preferably intravenous or subcutaneous administration.

Thus, the composition of the invention may be designed in any pharmaceutical form compatible with such administration routes, including but not limited to solutions, suspensions, dispersions, lyophilized powders, capsules and tablets, ointments, creams and gels, implants and devices and transdermal patches. Accordingly, the said composition can be formulated as emulsions, liposomes, polymers matrices, particles, or designed as a conjugate in which palytoxin (PLTX) is covalently bound to a carrier, or as any other formulation able to potentiate palytoxin anti-tumoral effect.

Accordingly, the composition may comprise one or several pharmaceutically acceptable excipients.

The expression “pharmaceutically acceptable” refers to compounds of pharmaceutical grade that are physiologically tolerable, can be metabolized and do not typically produce allergic or similar undesirable reactions, such as gastric upset, dizziness and the like when administered to a human. Preferably, as used herein, the expression “pharmaceutically acceptable” means approvable by a regulatory agency of the Federal or state government or listed in the U.S. Pharmacopeia or other generally recognized pharmacopeia for use in animals, and more particularly in humans. Such pharmaceutical excipients can be solvents or vehicles, such as water, saline, glycerol, ethanol and the like, as well as oils, including those of petroleum, animal, vegetable or synthetic origin, such as peanut oil, soybean oil, mineral oil, sesame oil and the like.

The term “carrier” refers to a carrier molecule to which palytoxin (PLTX) is (covalently) bound, and can be selected from a wide variety of known carriers encompassing, but not limited to, peptides, proteins, fatty acids, toxin or toxoids.

In the context of the invention, the expression “treatment of a subject suffering from a brain tumor”, as used herein, means the inhibition of the growth of the corresponding cancer cells. Preferably, such treatment also leads to the regression of tumor growth or the inhibition of metastasis spread in the brain. Most preferably, such treatment leads to the complete regression of the brain tumor.

As used herein, the term “preventing”, “prevention” generally means to avoid or minimize or delay the proliferation or dispersion of tumor cells within the subject, while the treatment of cancer can lead to the complete elimination of tumor cells. The term “prevention” encompasses reducing the likelihood of occurrence or reoccurrence brain metastasis.

Palytoxin (PLTX), an analogue or a derivative thereof is contained in said composition in an amount effective to achieve the intended purpose, and in dosages suitable for the chosen route of administration.

An “effective amount” is an amount which is sufficient to provide the desired effect, namely to stop the progression or to induce the regression of tumor growth or metastasis spread in the brain. The doses used for the administration can be adapted as a function of various parameters, in particular as a function of the mode of administration used, of the target pathology, or alternatively of the desired duration of treatment. Naturally, the form of the pharmaceutical composition, the route of administration, the dosage and the regimen depend on the condition to be treated, the severity of the illness, the age, weight, and sex of the subject, etc. The ranges of effective doses provided below are not intended to limit the invention and represent preferred dose ranges. However, the preferred dose can be tailored to the individual subject, as is understood and determinable by one of skill in the art, without undue experimentation.

As an illustration, an effective amount of PLTX is comprised between 0.1 ng/kg and 200 ng/kg, preferably between 1 ng/kg and 100 ng/kg, and most preferably between 5 ng/kg and 50 ng/kg. Dose level may request further refinement, potentially considering the case of conjugates for example, where the molecular weight and the activity of the conjugate thereof may influence it. The skilled artisan is readily credited with determining a suitable dosage that falls within the ranges, or if necessary, outside of the ranges.

Still preferably, said effective amount corresponds to a daily effective amount.

In a second aspect, the invention relates to a method for preventing and/or treating a subject suffering from a brain tumor comprising the administration to said subject of an effective amount of a composition comprising palytoxin (PLTX), an analogue or a derivative thereof, wherein said administration is a non-cerebral administration.

Yet, no limitation of the invention is intended by the details of the examples. Rather, the invention pertains to any embodiment which comprises details which are not explicitly mentioned in the examples herein, but which the skilled person finds without undue effort.

### EXAMPLES

### 1—In Vitro Anti-Tumoral Efficiency of Palytoxin obtained from Different Sources

The objective of this experiment was to test the survival of different primary or secondary brain cancer cell lines after treatment with PLTX obtained from various coral biomass.

Palytoxin was purified from known species belonging to distinct phylogenetic groups of Palythoa corresponding to Palythoa heliodiscus and Palythoa aff. clavata and from one undescribed species Palythoa sp. Pc001. (Known methods of extraction includes the ones disclosed in international patent application WO2015090591 or in RIOBO & FRANCO, Toxicon. Vol. 57(3), p:368-75. 2011).

A crude extract of Palythoa heliodiscus is also used. For obtaining this extract, corals polyps are cut off their support and then directly frozen and stored on dry ice. The frozen polyps are then crushed in a blender in purified water, slightly acidified with weak acid. The mixture is stirred for 1 h to 5 h then phase separation is done by centrifugation at 3000 g for 10 min. The supernatant is decanted from pelleted debris and then processed for biological testing.

These different palytoxin extracts were then tested at concentrations ranging from 10⁻⁴ to 10² pM, on selected brain tumor cell lines as listed in Table 1.

TABLE 1

| Cell | | | | Brain |
|---|---|---|---|---|
| lines | Origin | Primary site | Subtype | tumor |
| U-87 | Human | Central_nervous_system | Diffuse astrocytic and oligodendroglial | primary |
| MG | | | tumors/glioblastoma | |
| U-118 | Human | Central_nervous_system | Diffuse astrocytic and oligodendroglial | primary |
| | | | tumors/glioblastoma | |
| A-375 | Human | Skin | Melanoma | secondary |
| A 549 | Human | Lung | Metastatic non_small_cell_carcinoma | secondary |

Cells were grown according to the corresponding ATCC culture protocol. Equivalent dilution range of DMSO was used as negative control.

For survival assays, cells at passage 5 to 25 (p5-p25) were plated at 5.000 cell/well in 96 well plates, with immediate addition of PLTX to the media. Cell number and their viability were checked in triplicates at 72 h after drug treatment, using Resazurin (Alamarblue). Furthermore, cell cultures were checked for Mycoplasma contamination both before and at the end of experiments through PCR Mycoplasma Test Kit (PROMOKINE).

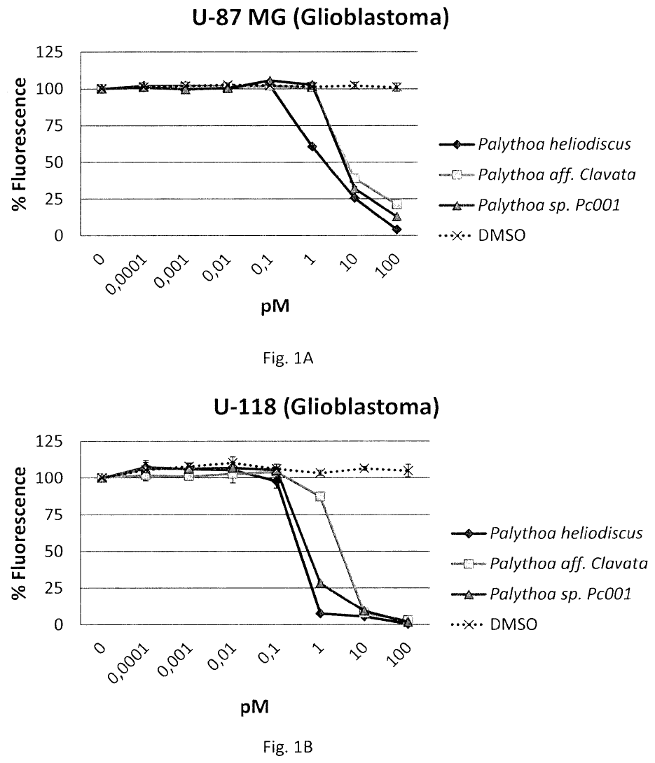

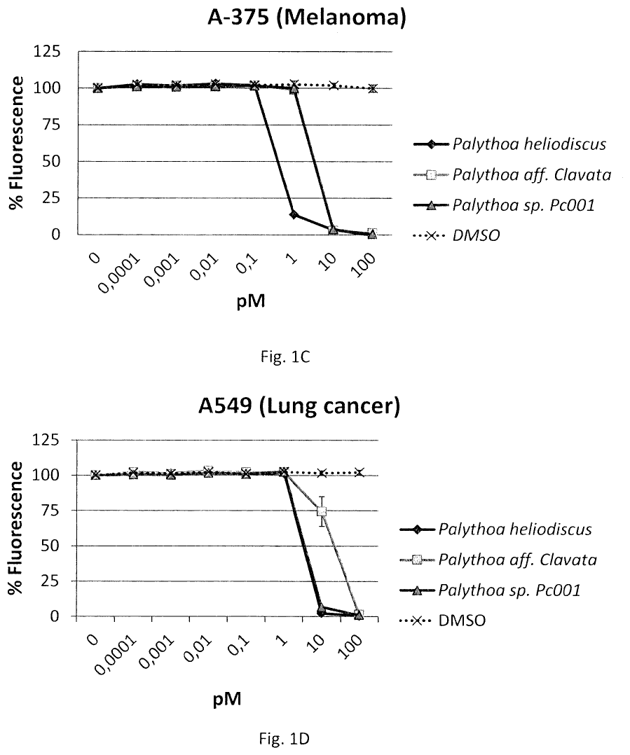

FIGS. 1A, 1B, 1C and 1D show the survival curves of U-87 MG, U-118, A-375 and A549 respectively, 72 h after treatment at increasing doses of Palytoxin purified from Palythoa heliodiscus, Palythoa aff Clavata and Palythoa sp. Pc001 (undisclosed results obtained with crude extract from Palythoa heliodiscus).

The results show that palytoxin from the 3 tested coral species, induces drastic cell mortality at low doses (<10 pM) independently of the phylogenetic group they belong to. Palytoxin obtained from Palythoa heliodiscus is shown to have a higher anti-cancer efficiency on cancer cell line survival. Interestingly, the surviving curves show that palytoxin induces a drastic switch from 100% survival to 0% survival within one log only (and within 2 logs for U-87 MG cell line), in accordance to its known cytotoxic activity. The results also evidence that PLTX is an efficient toxin against cell lines from glioblastoma, and highly metastatic melanoma and lung cancer.

### 2—In-Vivo Anti-Tumoral Effect of Palytoxin obtained from Different Sources

This two-stage experiment provides an in-vivo proof of concept of PLTX efficiency on tumor progression and metastatic invasion on a highly metastatic lung tumor, using the chicken embryo CAM assay (chorioallantoic membrane assay). This model also provides a fine and efficient way to compare PLTX extracted from different sources of corals.

Fertilized white Leghorn eggs were incubated at 37.5° C. with 50% relative humidity for 9 days. At this time (E9), the chorioallantoic membrane (CAM) was dropped by drilling a small hole through the eggshell into the air sac and a 1 cm² window was cut in the eggshell above the CAM.

The human lung cancer cell line A549 was cultivated in. F-12K medium with 10% FBS (and 1% penicillin/streptomycin). Cells (at 80% confluency, passage 21) were detached with trypsin, washed with complete medium and suspended in PBS.

Then, for tumor cell graft: an inoculum of 1.10⁶ cells was added onto the CAM of each egg (E9). Eggs were then randomized in 3 groups, of fourteen to eighteen eggs each, corresponding to treatment with PLTX from Palythoa Heliodiscus, Palythoa aff Clavata and Palythoa sp. Pc001.

At day 10 (E10), tumors began to be detectable and were treated during 10 days, every two days (E11, E13, E15, E17) by dropping onto the tumor 100 μl of vehicle (0.2% DMSO in PBS) or a Palytoxin at 1 nM purified from Palythoa heliodiscus or from Palythoa aff. Clavata and Palythoa sp. Pc001.

At day 18 (E18) the upper portion of the CAM was removed, washed in PBS and then directly transferred in PFA (fixation for 48 h) and the tumors were then carefully cut away from normal CAM tissue and weighted.

In parallel, a 1 cm² portion of the lower CAM was collected in 8 embryos per group to evaluate the number of metastasis cells. Genomic DNA is extracted from the CAM, and analyzed by qPCR with specific primers for Alu sequences. Metastasis is expressed as a ratio of QPCR signal of the different groups compared to group PLTX from Palythoa Heliodiscus.

Table 2 presents the tumor weight, relative quantity of metastasis and survival rate (SEM: Standard error of the mean).

TABLE 2

| | Palythoa | Palythoa aff. | Palythoa sp. |
|---|---|---|---|
| | Heliodiscus | Clavata | Pc001 |
| Tumor weight (mg) | 16.63 | 17.49 | 18.16 |
| SEM | 0.52 | 0.64 | 0.89 |
| Metastasis | 1 | 3.27 | 1.53 |
| SEM | 0.24 | 1.19 | 0.40 |
| % of embryo death | 11 | 5 | 26 |

Compared to untreated tumors (DMSO) having an average weight of 30 mg, the results confirm that palytoxin extracted from the 3 coral species efficiently reduces tumor size, said inhibition being stronger with the palytoxin purified from Palythoa Heliodiscus.

Moreover, the results show that the tumor growth inhibition by palytoxin (PLTX) is associated to metastasis inhibition, said metastasis inhibition being more profound with palytoxin (PLTX) purified from Palythoa Heliodiscus.

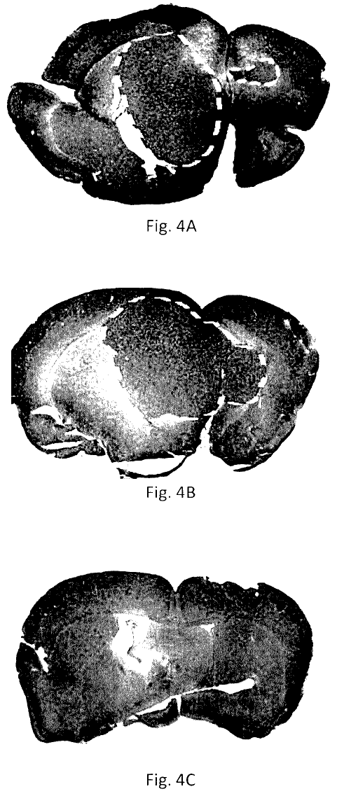

As the coral Palythoa Heliodiscus belongs to a phylogenetic group distinct from the 2 other corals Palythoa aff. Clavata and Palythoa sp. Pc001 (international patent application WO2015090591, FIG. 4), the ability to produce highly efficient anti-cancerous PLTX seems to be species independent, therefore opening the opportunity to extract PLTX from various biomass. This said, Palythoa sp. Pc001 showed the less efficient tumor inhibition and the highest lethality, being therefore the poorest coral candidate for therapeutic purpose.

As a second part of the experiment, in vivo toxicity of palytoxin from Palythoa Heliodiscus on chicken embryos was further investigated with increasing concentration of palytoxin (i.e. 2 nM, 20 nM, and 200 nM). Embryo-survival and sign of toxicity or developmental abnormalities were assessed after 10 days of treatment (same regimen as above) on twelve to eighteen embryos per group.

The list of the endpoints for toxicity is as follows:
Head: size, closure, eyes, ear, face and branchial arc derivatives, mobility;
Body: size, axis deformation, ventral and dorsal closures, caudal formation, sexual area;
Limbs: size, axis morphology, mobility;
Skin: appendage formation, attachment, blood vessel ;
Extra-Embryonic structures: vascularization, transparency, attachment, blood vessel.

The results are summarized in Table 3.

TABLE 3

| | | | List of abnormalities among | | | |
|---|---|---|---|---|---|---|
| | | | live embryos | | | |
| | | | | | | Extra- |
| | | Embryo | Head | Body | Limbs | emb |
| Treat- | N | survival | abnor- | abnor- | abnor- | abnor- |
| ment | (Embryos) | Rate | malities | malities | malities | malities |
| DMSO | 12 | 92% | 0/11 | 0/11 | 0/11 | 0/11 |
| PLTX | 17 | 100% | 0/17 | 0/17 | 0/17 | 0/17 |
| 2 nM | | | | | | |
| PLTX | 18 | 72% | 0/13 | 0/13 | 0/13 | 13/13 |
| 20 nM | | | | | | |
| PLTX | 15 | 7% | 0/1 | 0/1 | 0/1 | 1/1 |
| 200 nM | | | | | | |

The results show an acute toxicity at the dose of 200 nM: 93% of death after the last treatment. At 20 nM of palytoxin—i.e. 20 times the dose having a proven efficiency (1 nM) as seen in the first part of the experiment, the death ratio is lower (72%) and no abnormalities are seen in the embryos, while an alteration of the CAM was observed in all eggs of this group, said alteration being located at the treatment site. At the lowest dose (2 nM)—i.e. twice the efficient dose (1 nM), no deaths and no abnormalities among the 22 endpoints were detected in the embryos neither in the CAM, showing no adverse effect.

Based on the obtained results, it was concluded that PLTX is safe to be used in vivo against cancer when properly diluted. It was decided to use palytoxin from Palythoa Heliodiscus for the future experiments (methods of extraction according to Riobo & Franco, Toxicon. Vol. 57(3), p:368-75. 2011).

### 3 In Vivo Therapeutic Efficiency of Palytoxin on Primary Brain Tumor

This experiment uses a mouse model of highly invasive human glioblastoma U-87 MG cells, grafted in nude mouse brain to test the efficiency of PLTX on tumor growth and mouse survival.

For testing in vivo anti-tumoral effect of palytoxin, intracerebral (also named intraparenchymal) injection (0.5 ng total) of palytoxin was performed in the parenchyma located above the tumor on the day of tumor graft (group named PLTX intracerebral). As a negative control, a DMSO intracerebral injection was performed in the same location (group named DMSO) In addition, since palytoxin is known to be a potent toxin, and since mouse affected by a brain tumor may be weakened and more sensitive to toxins, palytoxin tolerance has been checked using intraperitoneal (IP) injection of PLTX (25 ng/kg, equivalent to 0.5 ng/mouse) twice a week, starting 5 days after tumor graft (group named PLTX intraperitoneal). No therapeutical benefit was expected for such treated IP mice, as palytoxin has never been reported for its ability to cross the BBB.

All experimental procedures using mouse were carried out according to a protocol approved by institutional review board and the French ethical committee. A total of 100 000 U-87 MG cells were stereoctically injected in the corpus callosum (+1 mm anterior to bregma, −1 mm lateral and −2 mm in deep of the cortex surface) of 22 six-week-old athymic nude mice under deep anesthesia as previously described in TCHOGHANDJIAN et al. (Brain Pathol., vol. 20: 211-221, 2010). Palytoxin intracerebral injections were performed in the cortex parenchyma above—and not inside—the tumor graft, in order to also assess potential parenchymal toxicity as well as anti-tumoral efficiency. To do so, the stereotaxic needle was elevated 0.2 mm higher and 0.5 ng of PLTX was injected after a 7 minute-break for brain tissue to return to their original position around the needle, to separate the PLTX and tumor cell injection site. Mice from PLTX intraperitoneal group were treated with palytoxin at 25 ng/kg (from 0.44 to 0.57 ng/mouse), starting 1 week after cell graft, twice a week. After surgery, animals were observed until they fully recovered from the experimental procedure and clinical signs were observed daily throughout the experiment.

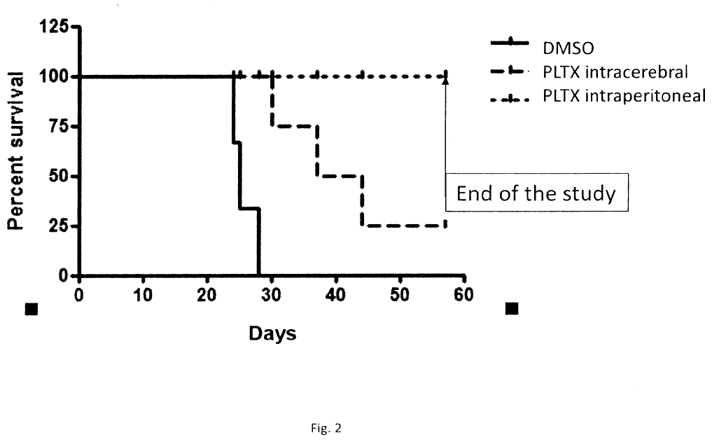

FIG. 2 shows the survival curves of mice treated with intracerebral DMSO injection on tumor graft day as negative control (DMSO, with intracerebral PLTX 0.5 ng injection on tumor graft day (PLTX intracerebral) and with intraperitoneal (IP) PLTX 25 ng/kg injection starting 5 days after tumor graft (PLTX intraperitoneal). The survival curve was determined using GRAPHPAD PRISM4 software.

Mice injected intracerebrally with 0.5 ng PLTX exhibited a highly significant survival prolongation of 173% compared to mice injected intracerebrally with DMSO (44.2±6.4 versus 25.5±0.8 days, p=0.0018), showing that PLTX helps preventing glioblastoma progression. Surprisingly, 30 days after all. DMSO mice died from glioblastoma progression, all mice injected IP with PLTX at 25 ng/kg were still alive: thus, they were voluntarily sacrificed for histological analysis, euthanasia representing the end of the corresponding survival curve. As an evidence, PLTX given as the IP 25 ng/kg treatment, starting 5 days after tumor graft, exhibits a highly significant survival prolongation of 224% compared to DMSO control group (57 versus 25.5±0.8 days, p=0.0018), and of at least 129% compared to the group PLTX intracerebral 0.5 ng (57 versus 44.2±6.4 days, p=0.0018).

In parallel, body weight and clinical status of mice were recorded daily during the experiments.

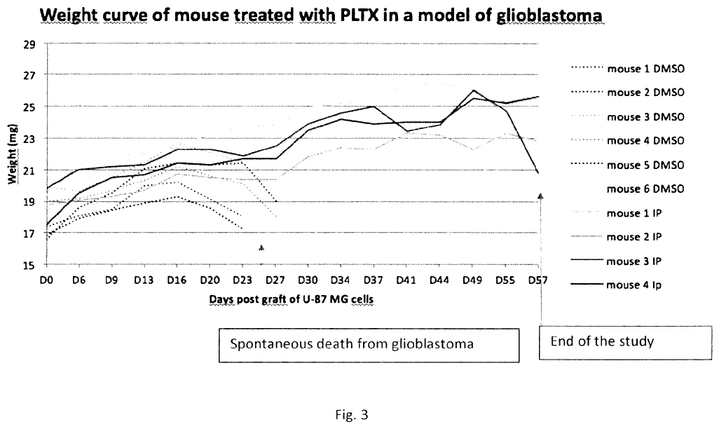

FIG. 3 shows the weight curve of mice treated with PLTX intraperitoneal injection (mouse 1 IP to mouse 4 IP), and with DMSO intracerebral injection (mouse 1 DMSO to mouse 6 DMSO), from the day of U-87 MG cells injection (DO).

Clinical signs that were observed in mice from both intracerebral groups (DMSO and 0.5 ng) were as follow: loss of weight together with loss of appetite, ataxia, prostration, altered alertness that are standard signs associated to glioblastoma growth. Except these typical glioblastoma clinical signs that led to mice euthanasia within 3 days, no other clinical signs that could be attributed to PLTX treatment were observed. At Day 57 post injection, mice from the PLTX IP group were still alive and have been voluntarily euthanatized. It is noticeable that at the time of euthanasia, 3 out of 4 mice showed absolutely no clinical signs, while one mouse appeared atonic and partially paralyzed, with symptoms suggesting PLTX toxicity accumulation due to 15 repetitive injections rather than glioblastoma progression. Brains have been sampled and fixed in formol 4% for subsequent histology examination. Brains appeared macroscopically normal.

As soon as severe clinical signs were detected, mice were anesthetized and PBS-perfused to allow for brain collection and fixation in formol 4%, followed by paraffin embedding according to standard procedures for subsequent histology (Hematoxylin and Eosin staining) and immunohistochemistry of (Ki67 (proliferation marker, VENTANA MEDICAL SYSTEMS, 1/100), caspase-3 cleavage (apoptosis maker, BD Biosciences, 1/500), GFAP (differentiation, astrocytic marker, VENTANA MEDICAL SYSTEMS, 1/500) and CD45,(total leucocytes, inflammation, 1/500). Staining were performed according to validated protocol as in TCHOGHANDJIAN et al. (Cell Death and Disease, vol. 7(8):e2325, 2016)

Hematoxylin and Eosin staining allowed visualizing and delineating the tumors. Tumors exhibit typical histological, size and morphology of glioma tumors as usually observed after U87MG intracranial injection.

FIGS. 4 shows a representative histological Hematoxylin and Eosin staining at the day of death for each group of mice: DMSO at 25 days of survival (FIG. 4A), PLTX intracerebral at 45 days of survival (FIG. 4B) and PLTX intraperitoneal at 60 days of survival (FIG. 4C) (scale bar=2.5 mm).

In the DMSO group, 6 out of 6 mice developed a tumor in just 25 days on average. In the PLTX intracerebral group, only 4 out of 5 animals also developed a tumor, and in 44 days on average. Those tumors were, similar to the DMSO group in term of histology, proliferation (43.3±10.85% and 52.5±2.5% ki67 positive cells in control and treated group respectively), differentiation (no GFAP Positive cells in tumors of both groups), and apoptosis (3±0.73% and 2.5±0.96% caspase-3 positive cells in control and treated group respectively). Since those animals showed an increased life expectancy, it can be concluded that a single PLTX local treatment significantly delays the tumor growth and related symptoms but is not sufficient to abolish tumor development and formation. Interestingly, one mouse (the longest survivor) didn't develop any tumor but instead a vimentin positive scare of gliosis, which could be a promising result of a tumoral regression due to the treatment in this animal.

In the PLTX intraperitoneal group, only 1 out of 4 animals developed a late tumor in 57 days. Another mouse exhibited only few isolated abnormal cancer cells. Those tumors showed low proliferation rate (17.5±7.5% Ki67 positive cells versus over 40% in the control and PLTX intracerebral groups). Most promisingly, two mice did not exhibit any tumor but instead a vimentine positive scar, suggesting a tumor development in 5 days before treatment following by a complete remission after PLTX intraperitoneal repeated treatment. These differences in tumor occurrence observed between DMO group and intraperitoneal group are statistically significant (p=0.033, Fisher Exact test).

Table 4 recapitulates mouse survival and tumor formation rate per group.

TABLE 4

| | Negative | PLTX | PLTX |
|---|---|---|---|
| | Control | intracerebral | Intraperitoneal |
| | (DMSO) | 0.5 ng | 25 ng/kg |
| Average survival time | 25.5 ± 0.8 days | 44.2 ± 6.4 days | 57 ± 0.0 days |
| Survival increased | / | 173% | 224% |
| compared to control | | | |
| Animals with tumor | 6/6 | 4/5 | 1/4 |
| formation | | | |
| % of animals with | 100% | 80% | 25% |
| tumor formation | | | |

Moreover, CD45 immunostaining was performed to investigate inflammation. No staining was found in the brain parenchyma around the tumor in the PLTX intracerebral and PLTX intraperitoneal groups showing that PLTX local injection and repetitive intraperitoneal treatment does not induce major brain inflammation. In addition, blood and organs were sampled (spleen, liver, pancreas, kidney, lung, heart and muscles) from 4 PLTX intraperitoneal mice to assess toxicity on selected organs and blood formulation. The results established that PLTX intraperitoneal injection did not alter the mice blood formulation (Red blood cell count, hemoglobin, hematocrit, reticulocyte, platelet and white blood cell counts), and did not show any sign of major inflammation, neither abnormal organ size or color at necropsy on 3 mice of the PLTX intraperitoneal group. The mouse showing sign of distress at euthanasia had normal blood counts and showed lightly darker organs due to poor perfusion induced by heart failure before perfusion. Finally, the results established that palytoxin injected into the peritoneum has been well tolerated and did not cause any lesion at the site of injection. Moreover, considering the DL50 of palytoxin injected intraperitoneally previously reported to range between 400 ng/kg and 720 ng/kg, the therapeutic window for PLTX treatment thus appears to be very large (see: MOORE et al., Nature, vol. 172, 495-498, 1971 ; LEVINE et al., Toxicon, vol. 25, p:1273-1282, 1.987; RHODES et al., N. Z. J. Mar. Freshw. Res. 36, 631-636, 2002).

To conclude, mice treated into the peritoneum with palytoxin showed efficient tumor destruction and survived much longer than the mice from the group receiving palytoxin intracerebrally (in the parenchyma directly above the tumor), therefore showing evidence that PLTX is able to cross the BBB to enter the brain and reach the tumor. This new and unexpected observation allows for the first time to treat brain tumor with PLTX injected outside the brain using parenteral or even enteral treatment, making treatment observance easier for patient. Moreover, it should be noticed that the palytoxin intraperitoneal treatment has been initiated 5 days after tumor cells implantation, which suggests that, the observed anti-tumoral effect is clearly a curative effect.

Therefore, palytoxin is shown to have a potent anti-tumoral action in vivo, with a broad therapeutic window (efficient a very low dose, way below toxic dose). The observed anti-tumoral effect obtained upon intraperitoneal injection indicates that PLTX is able to reach the brain by crossing the blood brain barrier.

Such efficient drug candidate for glioblastoma have never been reported before, even with molecules showing the best IC50 in vitro. With a 224% increased survival PLTX treatment is an unforeseen treatment hope. Comparatively, mice treatment by paclitaxel coupled to a peptide vector allowing blood brain barrier crossing led to only 15% improvement in survival rate in comparison with untreated mice (REGINA et al., British Journal of Pharmacology, Vol. 155(2), 185-197, 2008) while it is reported 40% increase for Bortezomib intracranial pump (WANG et al., Journal of Neurosurgery, Vol. 128(3):695-700, 2018); 23.6% for Juglone (natural cytotoxic pigment) and even only 160% for Temozolomide (standard of care, WU et al., BMC Neurology, Vol. 17, 70, 2017).

### 4 In Vivo Therapeutic Efficiency of Palytoxin on Secondary Brain Cancer

This experiment provides an in-vivo proof of concept of palytoxin (PLTX) efficiency on secondary brain cancer, meaning cancers originating in the other areas of the body that has spread to the brain by metastasis. Impact of PLTX on growth and brain invasion of the A549 highly metastatic human lung tumor is studied in the chicken embryo CAM (chorioallantoic membrane) assay as described above and compared to standard-of-care paclitaxel.

Table 5 presents the tumor weight, relative quantity of metastasis in low CAM, in chicken embryo brains and survival rate.

TABLE 5

| | Negative | | |
|---|---|---|---|
| | control | Positive control | |
| | (DMSO) | Paclitaxel 25 μM | PLTX 1 nM |
| Tumor weight (mg) | 25.431 ± 1.902 | 20.558 ± 1.902 | 19.346 ± 1.143 |
| | N = 17 | N = 16 | N = 16 |
| Metastasis relative amount, | 1 ± 0.409 | 0.404 ± 0.126 | 0.059 ± 0.043 |
| in low CAM | N = 7 | N = 7 | N = 8 |
| Metastasis relative amount, | 1 ± 0.238 | 0.390 ± 0.102 | 0.222 ± 0.056 |
| in embryo brain | N = 7 | N = 8 | N = 8 |
| % of embryo death | 23%, N = 22 | 24%, N = 21 | 24%, N = 21 |

The results confirmed that PLTX is significantly as efficient as paclitaxel to reduce tumor weight without influencing embryo survival. PLTX is significantly more efficient than paclitaxel to reduce metastases in the low CAM and in the brain of the chicken embryos.

Most specifically PLTX treatment at 1nM significantly reduced brain metastases amount in the brain of 78% compared to controls (0.01≥p value>0.001, N=7, One-way ANOVA with post t-tests) while paclitaxel showed a reduction of 61% only (0.05≥p value>0.01). PLTX is therefore 43% more efficient than paclitaxel on brain metastases amount.

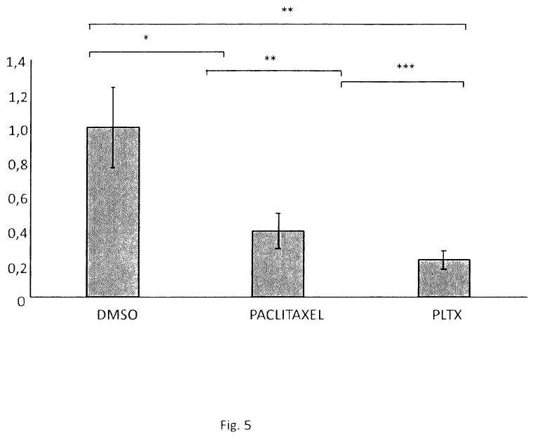

FIG. 5 shows relative metastasis invasion in brain of the chicken embryos at the end of the experiment (day E 19 of development) after standard-of-care (paclitaxel) and palytoxin 1 nM (PLTX) treatment, as reported to control treatment (DMSO). (Statistics: One way ANOVA; one star (*): 0.05≥p value>0.01; Two stars (**): 0.01≥p value>0.001; Three stars (***): 0.001≥p value)

Those results show that PLTX can efficiently target brain metastases issued from human lung cancer despite blood-brain barrier and brain micro-environment that usually lower most drug efficiency in the brain.

### 5 Anti-Tumoral Effect of Palytoxin (PLTX) on Explants of Human Brain Tumor Biopsies

To further investigate the anti-tumoral potential of PLTX, the physiological effect of PLTX on human 3D brain tumors is studied, using glioblastoma patient tumors, grown as explants. Most interestingly, this ex vivo model greatly facilitates the retention of native tissue architecture and brain micro-environment, which reflects better in vivo situation. Indeed, it is a 3 dimensional cell culture which maintains tumor cell-cell and cell-matrix interactions and includes cellular and molecular factors from the microenvironment, known to promote tumor cell proliferation, survival and invasion and therefore to inhibit drugs efficiency in the brain parenchyma (R. LANGLEY et al. Clinical Chemistry 59:1, 180-189 2013).

Tumors were obtained from patients requiring surgical intervention for therapeutic purpose and having given voluntary informed consent. Only glioblastoma samples with a validated histological diagnosis were used and patient did not receive any chemotherapy or radiotherapy before tumor resection. Tumors are cut into small pieces (500 μm thick are cultured on poly-lysine coated plates in DMEM, 10% heat-inactivated fetal bovine serum, 1% Penicillin/Streptomycin, 1% Na⁺/Pyruvate, 0.4% methylcellulose and appropriate antibiotics and growth factors. Explants are then treated with PLTX at 1 and 10 pM or with DMSO (vehicle), as negative control Explant growth and survival are monitored after 3 days.

Pictures of individual explants were taken before treatment (Day 0) and at the end of the experiment (Day 3). On each picture, explant core surface and cell migration front surface were measured and the ratio (D3/D0) are calculated. Those data were then treated via Mann & Whitney statistical test using the biostaTGV from French Jussieu University.

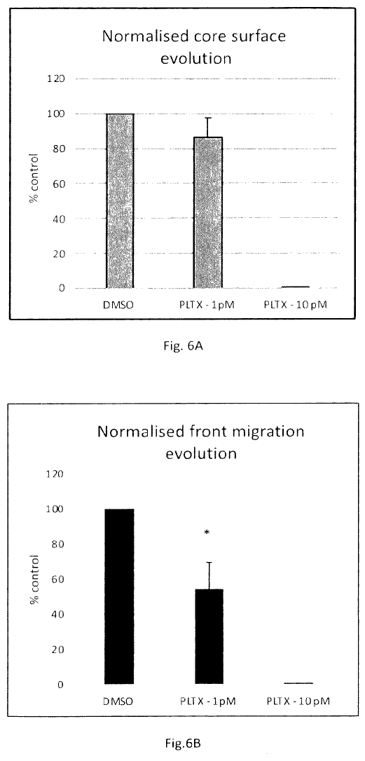

FIGS. 6 shows the quantification of explant core surface (A) and front migration (B) evolution within 3 days (D3/D0) after PLTX treatment at 1 and 10 pM expressed in percentage of control (DMSO), (star represents significativity with pvalue<0.05, Mann & Whitney).

PLTX treatment at 1 pM for 3 days induces overall a reduction of migration front of about 60% compared to control (N=5 patients, 2-9 explants each, significantly different distribution in Mann & Whitney test with pvalue=0.028) and of explant core size of 14% compared to control (N=5 patients, 2-9 explants each, Wilcoxon, pvalue=0.11). Those data show a drastic explant growth inhibition despite patient heterogeneity.

At 10 pM, all explants died integrally and detached (N=2 patients with 3 explants each).

PLTX therefore shows promising cytotoxic effects on ex vivo human glioblastomas, despite the micro-environment present in explant 3D cultures and the known patient heterogeneity. Those results foster great hopes for clinical application of the discovery.

### 6 Anti-Tumoral Action of PLTX on Primary and Secondary Brain Tumor Cell Lines

To broaden the spectrum of indications, effect of palytoxin on cell viability (IC50 assay) was tested on large set of brain tumor cell lines as listed in Table 6. For each brain tumor, cell lines are mentioned as “primary tumor” when cell line is a model of resident CNS cancer inside the BBB; or “Brain secondary tumor” if the cell line was derived from a brain metastasis in the original patient; or as “Brain metastasis” if brain metastasis has been documented in the literature after orthotopic or subcutaneous transplantation into mice or chicken embryo; or as “Brain metastasis risk” if cell line is metastatic but brain metastasis location not yet documented to our knowledge. (*refers to undisclosed data).

TABLE 6

| | | | PLTX |
|---|---|---|---|
| | | | IC50 at |
| Cell lines | Disease | Brain tumor | 72 h (pM) |
| A 375 | Melanoma | Brain metastasis | 0.1-1 |
| A 549 | Lung cancer | Brain metastasis | 01-10 |
| A-172 | Glioblastoma | Primary tumor | * |
| ACHN | Kidney carcinoma | — | 0.1-1 |
| BE(2), SK-N-BE clone | Neuroblastoma | Primary tumor | * |
| BT 20 | Breast cancer | Brain metastasis risk | 1-10 |
| BXPC3 | Pancreatic cancer | — | 01-10 |
| Calu-6 | Lung carcinoma | — | 0.1-1 |
| CHLA-01 | Medulloblastome | Primary tumor | * |
| CHLA-02-ATRT | Atypical Teratoid Rhabdoid Tumor | Primary tumor | * |
| CHLA-04-ATRT | Atypical Teratoid Rhabdoid Tumor | Primary tumor | * |
| CHLA-06-ATRT | Embryonic Atypical Teratoid Rhabdoid Tumor | Primary tumor | * |
| D458 Med | Medulloblastoma | Primary tumor | * |
| HDMB03 | Medulloblastoma | Primary tumor | * |
| Daoy | Desmoplastic Cerebellar Medulloblastoma | Primary tumor | * |
| DBTRG-05MG | Glioblastoma | Primary tumor | * |
| DLD-1 | Colorectal carcinoma | Brain metastasis risk | 01-10 |
| H1299 | Metastatic Lung NSCLC | Brain metastasis risk | 01-10 |
| HCT-116 | Metastatic colon carcinoma | Brain metastasis risk | 01-10 |
| H9 (HUT 78) | Cutaneous T lymphoma | Brain metastasis risk | 0.1-1 |
| IMR-32 | Neuroblastoma (abdominal metastasis) | Primary tumor | * |
| Karpas-299 | Lymphoma | — | 01-10 |
| LN-229 | Glioblastoma | Primary tumor | * |
| MDA-MB-231 | Metastastic breast adenocarcinoma | Brain metastasis risk | * |
| MDA-MB-361 | Brain metastasis of breast | Brain secondary tumor | * |
| | adenocarinoma | | |
| MeWO | Melanoma | — | 0.1-1 |
| NCI-H1915 | Brain metastasis from poorly | Brain secondary tumor | * |
| | Differentiated Lung Carcinoma | | |
| Panc-1 | Pancreatic cancer | — | 0.1-1 |
| PC-3 | Prostate cancer | Brain metastasis risk | 01-10 |
| PFSK-1 | Malignant Primitive | Primary tumor | * |
| | Neuroectodermal Tumor | | |
| Rec-1 | Lymphoma | — | 0.1-1 |
| SF767 | Glioblastoma | Primary tumor | * |
| sh-sy5y | Bone Marrow Neuroblastoma | Primary tumor | * |
| SK-N-SH | Bone Marrow Neuroblastoma | Primary tumor | * |
| SK-PN-DW | Metastatic peritoneal Primitive | Brain metastasis risk | * |
| | Neuroectodermal Tumor | | |
| SW 1088 | Astrocytoma | Primary tumor | * |
| U-118 | Glioblastoma | Primary tumor | 0.1-1 |
| U-373 MG | Glioblastoma astrocytoma | Primary tumor | * |
| U-87 MG | Glioblastoma | Primary tumor | 01-10 |

Cells were grown according to the corresponding ATCC culture protocol.

The cell number and their viability were determined after palytoxin treatment, using MTT, MTS or CellTiterGlow.

The results show that PLTX has a strong antitumoral effect on the brain tumor cell lines tested, including primary brain cancer and numerous other cancers able to metastase in the brain.

### 7 Palytoxin Blood Brain Barrier Crossing

In this experiment, crossing of the BBB by palytoxin and palytoxin analogs is tested in vitro and in vivo:

The in vitro testing is done on a rat syngeneic model using co-cultures of primary rat brain endothelial cells.

Primary cultures of brain endothelial cells, were prepared from 5- to 6-week-old Wistar rats by enzymatic and mechanical dissociation and purification as established by MOLINO et al., (Setting-up an In Vitro Model of Rat Blood-brain Barrier (BBB): A Focus on BBB Impermeability and Receptor-mediated Transport, J. Vis. Exp. (88), e51278, doi:10.3791/51278, 2014). Endothelial cells are seeded in the luminal compartment of coated six-well plate polyethylene insert filters (pore size 1.0 μm), to establish the endothelial cell monolayers. Astrocytes, prepared from neonatal Wistar rats, were seeded in the bottom of the six-well plates for co-culture in endothelial cell media containing DMEM/F12 supplemented with 20% bovine platelet poor plasma derived serum (ALFA AESAR), basic fibroblast growth factor 2 ng/mL, heparin 100 μg/mL, gentamycin 50 μg/mL, HEPES 2.5 mM, and hydrocortisone 500 nM. Under these conditions, the endothelial cells monolayers differentiate, express junction-related proteins and remain optimally differentiated. The endothelial layer integrity is checked by permeability coefficients (Pe) for lucifer yellow (LY). PLTX is added to the upper chamber and detected in the lower chamber to measure passive diffusion or active transport trough the endothelial layer on the filter.

The in vivo testing is done on adult animals (mice, rat and/or rabbit).

PLTX and analogs were administered intraperitoneally and intravenously at 10 and 25 ng/kg to animals.

After 12-24 h, animals were anesthetized, cerebro-spinal fluid was sampled, mice were perfused and brain collected for dissection. PLTX is detected on sample using high performance liquid chromatography coupled with mass-spectrometry, SPR or ELISA assay.

(undisclosed data)

1-15. (canceled)

16. A method for treating a subject suffering from a brain tumor comprising administering to said subject an effective amount of a composition comprising ply-toxin (PLTX.).

17. The method of claim 16, comprising a non-intracerebral administration.

18. The method of claim 16, wherein the treated subject is adult or juvenile.

19. The method of claim 16, further comprising administering another anti-tumoral agent to said subject.

20. The method of claim 19, wherein the another anti-tumoral agent is selected from the group consisting of temozolomide, carmustine and cytarabine.

21. The method of claim 16. wherein the subject suffers from a primary brain tumor.

22. The method of claim 16, wherein t le subject suffers from a secondary brain tumor.

23. The method of claim 21, wherein said primary brain tumor is selected from the group consisting of diffuse astrocytic and oligodendroglial tumors and embryonal tumors.

24. The method of claim 21, wherein said primary brain tumor is glioblastoma.

25. The method of claim 22, wherein the secondary brain tumor is selected from the group consisting of brain metastasis issued from lung tumors, breast tumors, melanomas, digestive tract tumors, renal tumors, lymphomas, pancreatic and prostate tumors.

26. The method of claim 25, wherein said secondary brain tumor is selected from the group consisting of brain metastasis issued from lung tumors, breast tumors and melanomas.

27. The method of claim 16, wherein said non-intracerebral administration is selected from the group consisting of oral, mucosal, intravenous, intramuscular, subcutaneous, intraperitoneal, intradermal and transdermal administrations.

28. The method of claim 27, wherein said non-intracerebral administration is an intravenous or a subcutaneous administration.

29. The method of claim 16, wherein the effective amount of palytoxin is comprised between 0.1 ng/kg and 200 ng/kg.

30. The method of claim 16, wherein the effective amount of palytoxin is comprised between 1 ng/kg and 100 ng/kg.

31. The method of claim 16, wherein the effective amount of palytoxin is comprised between 10 ng/kg and 50 ng/kg.

32. The method of claim 16, wherein palytoxin (PLTX) comprises a coral palytoxin.

33. The method of claim 32, wherein the coral is a Palpthoa species.

34. The method of claim 16, wherein palytoxin (PLTX) comprises a Palythoa Heliodiscus palytoxin.
